(19) United States
(12) Patent Application Publication
(10) Pub. No.: US 2012/0065554 A1
(43) Pub. Date: Mar. 15, 2012

(54) Dual Balloon Ablation Catheter with Vessel Deformation Arrangement for Renal Nerve Ablation

(76) Inventor: Michael Pikus, Golden Valley, MN (US)

(21) Appl. No.: 13/228,195

(22) Filed: Sep. 8, 2011

Related U.S. Application Data
(60) Provisional application No. 61/381,263, filed on Sep. 9, 2010.

(51) Int. Cl. A61B 18/14; A61B 18/02; A61N 7/00; A61B 18/12

(52) U.S. Cl. 601/2; 606/41; 606/21

(57) ABSTRACT

A balloon arrangement provided at a distal end of the catheter is configured for deployment within a vessel and comprises a distal balloon and a proximal balloon spaced apart from the distal balloon. An ablation arrangement is provided at the distal end of the catheter and configured to ablate target tissue proximate the vessel. A vessel deformation arrangement comprises at least a portion of the balloon arrangement and is configured to draw a wall segment of the vessel and the target tissue inwardly towards the ablation arrangement. The vessel deformation arrangement may include a port provided in a section of the shaft between the distal and proximal balloons. The port and balloon arrangement are configured to cooperatively draw the wall segment of the vessel and the target tissue inwardly towards the ablation arrangement in response to a negative pressure developed at the port.

### RELATED APPLICATIONS

This application claims the benefit of Provisional Patent Application Ser. No. 61/381,263 filed Sep. 9, 2010, to which priority is claimed pursuant to 35 U.S.C. §119(e) and which is hereby incorporated herein by reference in its entirety.

### SUMMARY

Embodiments of the disclosure are directed to apparatuses and methods for ablating target tissue from within a vessel. Embodiments of the disclosure are directed to apparatuses and methods for urging a vessel wall inwardly into proximity or contact with an ablation arrangement positioned within the vessel and delivering ablation therapy to target tissue. Embodiments of the disclosure are directed to apparatuses and methods for urging a wall of a renal artery inwardly into proximity or contact with an ablation arrangement positioned within the renal artery and delivering ablation therapy to perivascular renal nerves for the treatment of hypertension.

In accordance with various embodiments, an apparatus includes a catheter and a balloon arrangement provided at a distal end of the catheter. The balloon arrangement is configured for deployment within a vessel and comprises a distal balloon and a proximal balloon spaced apart from the distal balloon. An ablation arrangement is provided at the distal end of the catheter and configured to ablate target tissue proximate the vessel. A vessel deformation arrangement comprises at least a portion of the balloon arrangement and is configured to draw a wall segment of the vessel and the target tissue inwardly towards the ablation arrangement. In some embodiments, the vessel deformation arrangement comprises a port provided in a section of the shaft between the distal and proximal balloons and is fluidly coupled to a vacuum lumen of the catheter. The port and balloon arrangement are configured to cooperatively draw the wall segment of the vessel and the target tissue inwardly towards the ablation arrangement in response to a negative pressure developed at the port. With the vessel wall segment and target tissue inwardly drawn into contact or proximity with the ablation arrangement, the ablation arrangement is configured to deliver an ablation therapy to the target tissue adjacent a circumferential region of the vessel wall segment.

In some embodiments, less than a full 360° region of the target tissue can be subject to ablation. In other embodiments, the balloon and vessel deformation arrangements may be moved axially forward or backward relative to an initial treatment position within the vessel, allowing for ablation therapy delivery to a number of different axially and/or circumferentially spaced regions of the target tissue. In further embodiments, a multiplicity of balloon and vessel deformation arrangements may be provided at a distal end of a catheter shaft, each treating a different axial and/or circumferential region of the adjacent target tissue. Embodiments that involve repositioning of a single balloon and vessel deformation arrangement or deployment of a multiplicity of balloon and vessel deformation arrangements are directed to ablating target tissue proximate to longer vessels and structures of the body (e.g., vessels longer than a renal artery).

According to other embodiments, a catheter includes a flexible shaft having a proximal end, a distal end, a length, and a lumen arrangement extending between the proximal and distal ends. The length of the shaft is sufficient to access a patient's renal artery relative to a percutaneous access location. A balloon arrangement is provided at the distal end of the catheter and configured for deployment within the renal artery. The balloon arrangement is fluidly coupled to the lumen arrangement and comprises a distal balloon and a proximal balloon spaced apart from the distal balloon. An ablation arrangement is provided at the distal end of the catheter and configured to ablate perivascular renal nerves adjacent the renal artery. A port is provided in a section of the shaft between the first and second balloons and fluidly coupled to the lumen arrangement. The port and the balloon arrangement are configured to cooperatively draw a wall segment of the renal artery and perivascular renal nerves adjacent the renal artery wall segment inwardly into proximity or abutment with the ablation arrangement in response to a negative pressure developed at the port. The ablation arrangement may then be activated to ablate the perivascular renal nerves.

Method embodiments involve drawing a wall segment of a vessel and target tissue adjacent the vessel inwardly towards an ablation arrangement deployed in the vessel and, with the vessel wall segment and target tissue inwardly drawn into contact or proximity with the ablation arrangement, ablating the target tissue. Method embodiments also involve terminating ablation of the target tissue and drawing of the vessel wall segment, thereby allowing the diameter of the vessel wall segment to return to its original size. The vessel may be a renal artery of a patient, and the target tissue may include perivascular renal nerves adjacent the renal artery.

The ablation arrangement can include one or a combination of a cryothermal, electrical, acoustic, optical, mechanical, lysic, neurotoxin, or pharmacological ablation apparatuses, for example.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 illustrates a vessel deformation arrangement and an integral cryoablation arrangement in accordance with various embodiments;

FIGS. 5-8 illustrate several vessel deformation arrangements and integral high frequency AC ablation arrangements in accordance with various embodiments; and

FIG. 9 shows a representative renal ablation apparatus in accordance with various embodiments of the disclosure.

### DETAILED DESCRIPTION

In accordance with various embodiments, an intravascular apparatus includes a vessel deformation arrangement configured to urge a portion of the renal artery inwards, drawing the perivascular renal nerves inward as well. Deforming the portion of the renal artery inwards is preferably accomplished using a negative pressure drop (e.g., suction) produced by the intravascular apparatus. Continued suction draws the artery wall and adjacent perivascular renal nerve tissue inward, causing a narrowed or flattened arterial structure. With the target nerve tissue drawn into contact or proximity with the intravascular apparatus, a denervation therapy is delivered. Various types of ablation arrangements may be incorporated as part of the intravascular apparatus, including electrical, thermal, acoustic, mechanical, optical, lysic, and pharmacological ablation arrangements, for example, or combinations of these arrangements. Upon completion of the denervation therapy, the suction is removed and the diameter of the isolated renal artery segment resumes its original size.

Embodiments of the disclosure provide for effective ablation of perivascular renal nerves while significantly reducing the risk of thermal injury to the renal artery wall. Embodiments of an intravascular ablation apparatus employ a vessel deformation arrangement to draw perivascular renal nerve tissue into close proximity with an ablation arrangement. Because the vessel deformation arrangement reduces the distance between the target tissue and the ablation arrangement, effective ablation of the target tissue can be achieved using less ablative energy than conventional ablation approaches. This reduction in ablative energy required to denervate renal artery tissue, for example, results in a concomitant reduction in thermal injury to the renal artery wall. This reduction in ablative energy required to denervate renal artery tissue also reduces the amount of heat to be removed from the artery wall proximate the ablation arrangement, thereby reducing the complexity of (or need for) a cooling arrangement for ablation approaches that involve elevated temperatures at or near the artery wall (e.g., RF ablation).

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

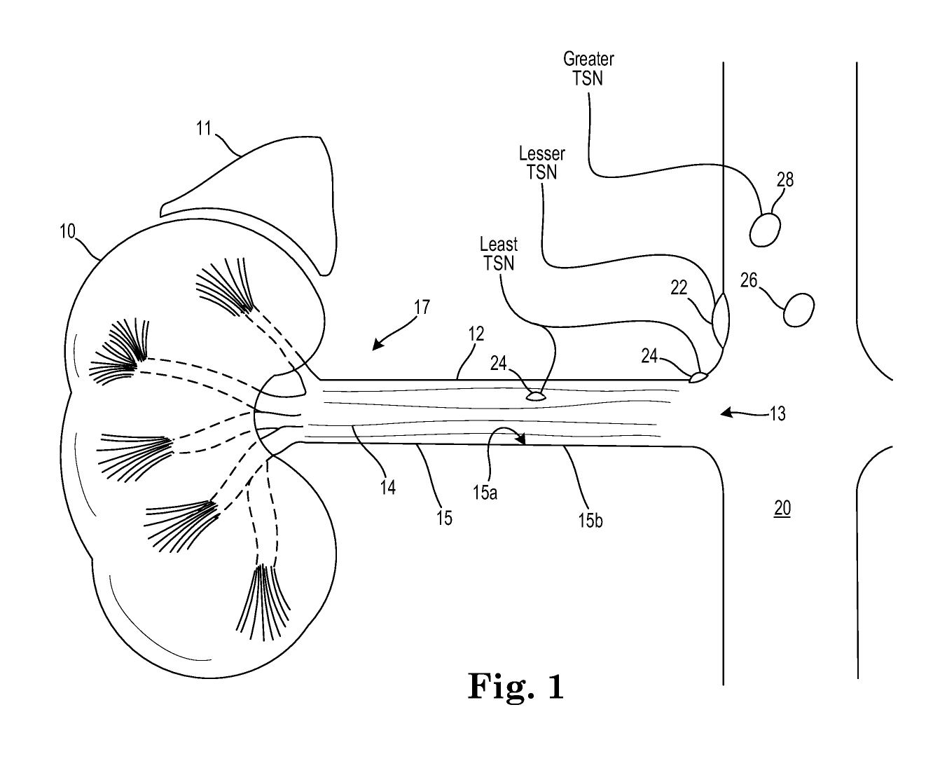

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

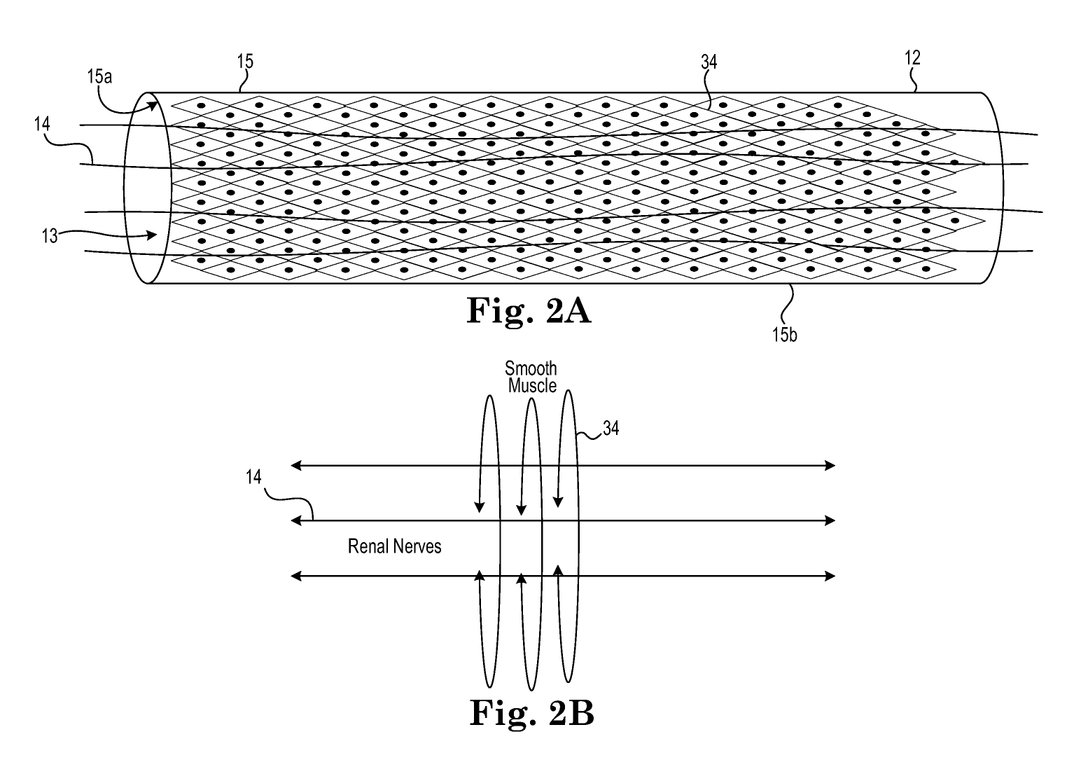

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

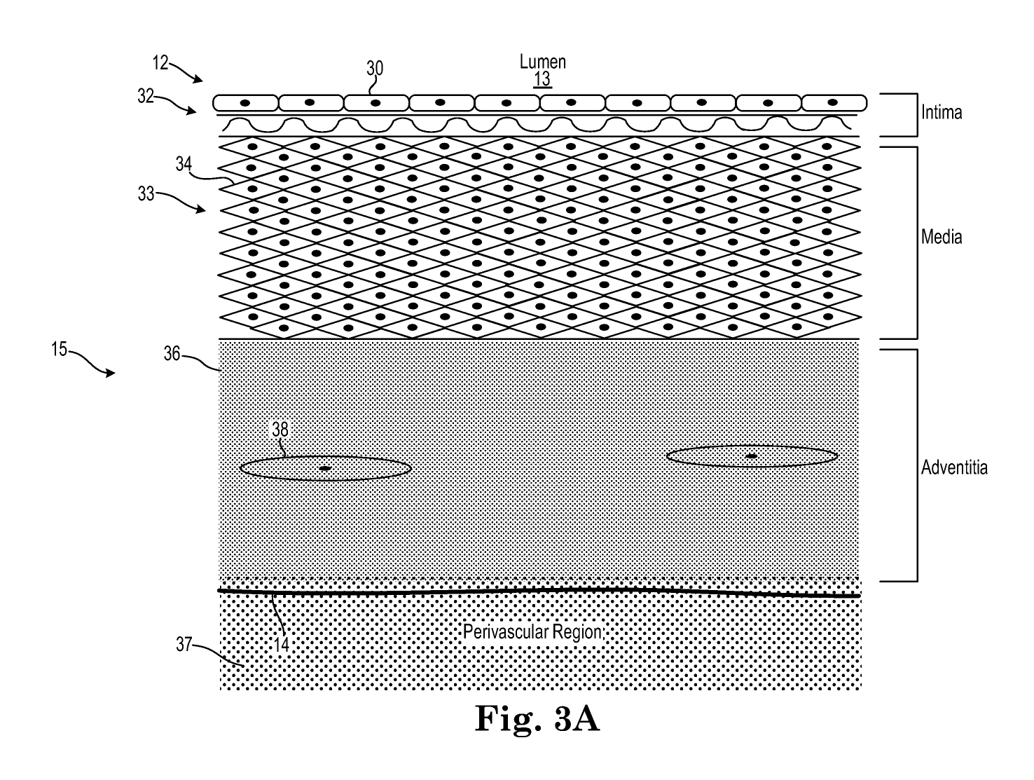

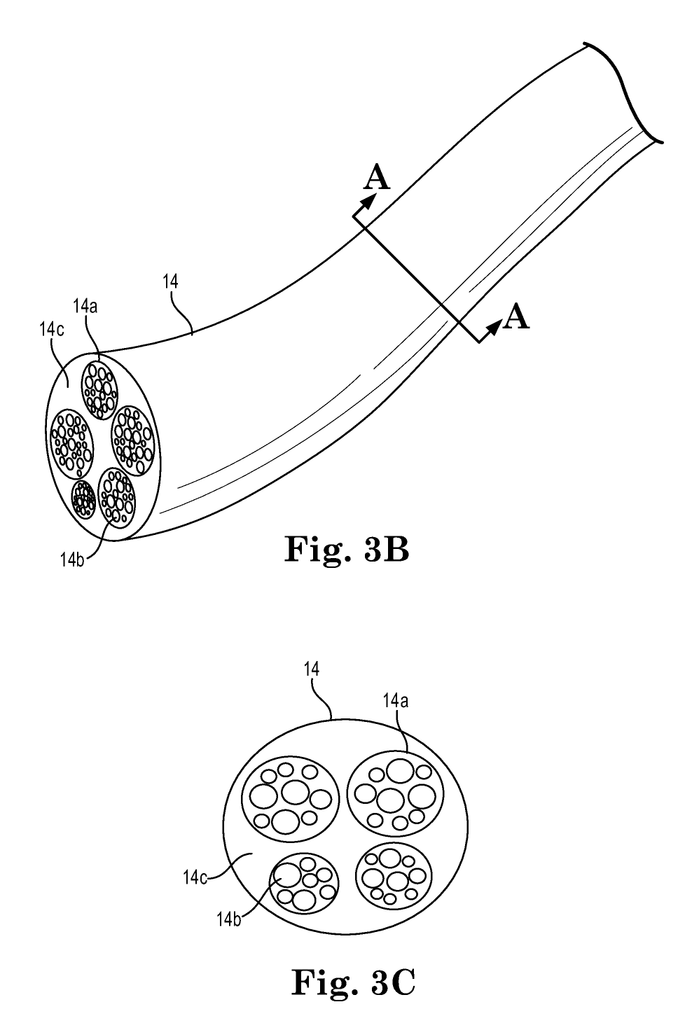

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b is preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

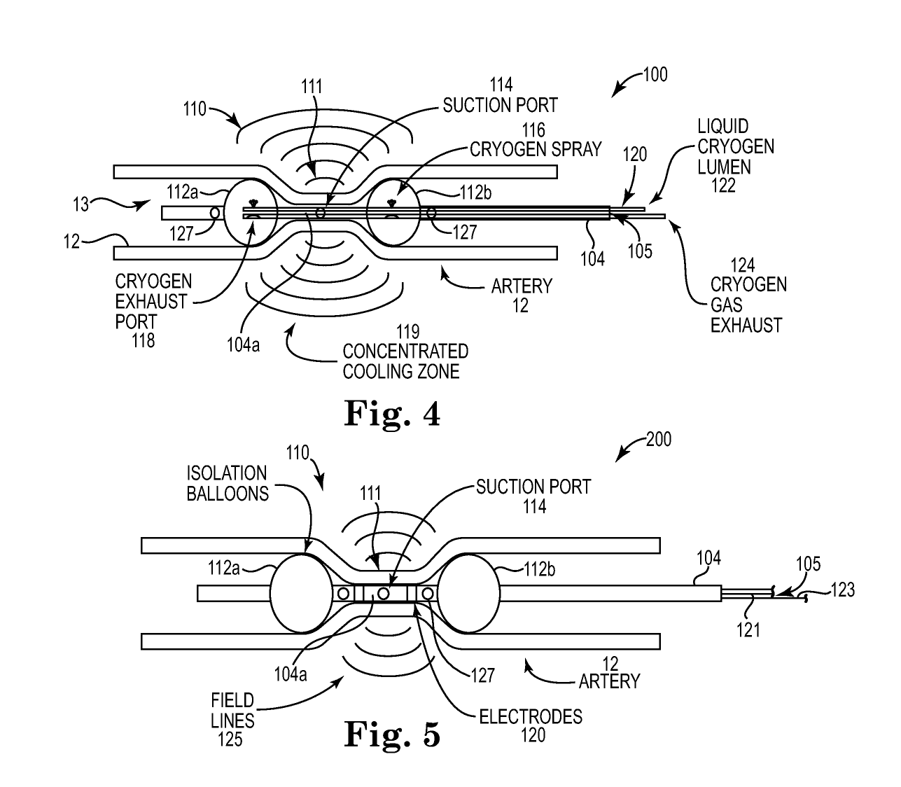

Turning now to FIG. 4, there is illustrated a distal end portion of a catheter 100 which includes a treatment apparatus 110 deployed within a vessel of the body in accordance with various embodiments. In the embodiment shown in FIG. 4, the treatment apparatus 110 of the catheter 100 is deployed in a patient's renal artery 12. The catheter 100 includes a flexible shaft 104 having a proximal end, a distal end, a length, and a lumen arrangement 120 extending between the proximal and distal ends. The length of the shaft 104 is sufficient to access the patient's renal artery 12 relative to a percutaneous access location. The treatment apparatus 110 includes a vessel deformation arrangement 111 which is fluidly coupled to the lumen arrangement 120 of the shaft 104.

The vessel deformation arrangement 111 includes a distal balloon 112a and a proximal balloon 112b. The distal and proximal balloons 112a and 112b are situated on the catheter shaft 104 in a spaced-apart relationship and fluidly coupled to the lumen arrangement 120. The distal and proximal balloons 112a and 112b re preferably separated from one another by a distance of about 4 mm to about 6 mm.

Each of the distal and proximal balloons 112a and 112b is fluidly coupled to a supply lumen 122 and an exhaust lumen 124 of the lumen arrangement 120. Inflation and deflation of the distal and proximal balloons 112a and 112b can be controlled by adjusting the pressures in the supply and exhaust lumens 122 and 124. A section 104a of the catheter shaft 104 between the balloons 112a and 112b includes a suction port 114 which is fluidly coupled to a vacuum lumen 105 of the lumen arrangement 120, which may be the main lumen of the shaft 104 or a separate lumen similar to the supply or exhaust lumen 122, 124.

After positioning the treatment apparatus 110 at a desired location within the artery 12, the vessel deformation arrangement 111 can be activated to draw a segment of the renal artery 12 into close proximity or abutment with the ablation arrangement of the treatment apparatus 110. As is illustrated in FIG. 4, the balloons 112a and 112b can be inflated to isolate a segment of the renal artery 12. A negative pressure generated at a proximal end of the vacuum lumen 105 produces a suction at the suction port 114, which is used to remove blood trapped in the isolated segment of the renal artery 12. Continued suction at the suction port 114 draws the wall of the renal artery 12 along the isolated segment inwardly and into contact with the section 104a of the catheter shaft 104 between the balloons 112a and 112b, causing a narrowed or flattened structure. Drawing the renal artery wall inwardly as shown in FIG. 4 advantageously draws the perivascular renal nerve tissue into close proximity with the ablation arrangement of the treatment arrangement 110. Upon completion of the denervation therapy, the suction is removed and the diameter of the isolated renal artery segment resumes its original size. The intravascular apparatus and catheter arrangement are then removed from the patient.

According to the embodiment shown in FIG. 4, the balloons 112a and 112b are configured as cyroballoons. Each of the distal and proximal balloons 112a and 112b is fluidly coupled to a supply lumen 122 and an exhaust lumen 124 of the lumen arrangement 120. According to some embodiments, the dual-balloon arrangement 112a and 112b is configured to deliver cryotherapy via phase-change cryothermal cooling. A liquid cryogen is delivered to the balloons 112a and 112b via the supply lumen 122. When released inside the balloons 112a and 112b via supply ports 116, the liquid cryogen undergoes a phase change that cools the balloons 112a and 112b by absorbing the latent heat of vaporization from surrounding tissue, and by cooling of the vaporized gas as it enters a region of lower pressure inside the balloons 112a and 112b (via the Joule-Thomson effect).

As a result of the phase change and the Joule-Thompson effect, heat is extracted from the surroundings of the balloons 112a and 112b, thereby cooling the adjacent section of the renal artery 12 and perivascular nerve tissue to a temperature below freezing within a concentrated cooling zone 119. The size of the zone 119 of concentrated cooling can be adjusted as needed by adjusting various aspects of the treatment apparatus 110, such as the spacing between the balloons 112a and 112b, the size and shape of the balloons 112a and 112b, the diameter of the catheter section between the balloons 112a and 112b, the type of cryogen used, and the temperature at which cryothermal ablation is conducted, for example.

The gas released inside the balloons 112a and 112b is exhausted through exhaust ports 118 and a return lumen 121 provided in the lumen arrangement 120 of shaft 104. The pressure inside the balloons 112a and 112b may be controlled by regulating one or both of a rate at which cryogen is delivered and a rate at which the exhaust gas is extracted. One or more temperature sensors 127 may be situated proximate or on the balloons 112a and 112b for measuring arterial tissue temperature. The lumen arrangement 120 of the catheter shaft 104 includes one or more conductors for coupling each of the temperature sensors 127 to an external temperature unit.

According to other embodiments, the treatment arrangement 110 includes a fluid transport mechanism configured to circulate a cryogen fluid through the section of the catheter shaft 104 situated between the balloons 112a and 112b. The treatment arrangement 110 can incorporate a cryotube, for example, fluidly coupled to the supply and exhaust lumens 122 and 124. A cryogen can be circulated through the cryotube via a hydraulic circuit that includes an external cryogen source, supply and return lumens 122, 124, and the cryotube. In the embodiments that incorporate a cryotube or similar cryogen circulation heat exchange element, the balloons 112a and 112b can be pressurized using an inert gas, for example.

Various embodiments disclosed herein may incorporate selected balloon, catheter, lumen, control, and other features of the devices disclosed in the following commonly owned U.S. patents and published patent applications: U.S. Patent Publication Nos. 2009/0299356, 2009/0299355, 2009/0287202, 2009/0281533, 2009/0209951, 2009/0209949, 2009/0171333, 2008/0312644, 2008/0208182, 2008/0058791 and 2005/0197668, and U.S. Pat. Nos. 5,868,735, 6,290,696, 6,648,878, 6,666,858, 6,709431, 6,929,639, 6,989,009, 7,022,120, 7101,368, 7,172,589, 7,189,227, and 7,220,257, all of which are incorporated herein by reference.

When using cryothermal energy devices placed in the renal artery 12 for ablation of perivascular renal nerves for treatment of hypertension, the greatest cooling is adjacent to the device. In order to achieve tissue temperatures for effective ablation of the renal nerves, other non-target tissues, such as the renal artery 12, are often injured as well. The representative embodiment shown in FIG. 4 provides an improved approach for effectively freezing the target tissue while reducing injury to the renal artery 12. A similar approach can be used in the renal veins, or a combination of renal artery 12 and renal vein. Other vessels (e.g., veins and other vasculature) can be treated using apparatuses and methods disclosed herein for similar drawing in of target tissue, such as to treat tumors or benign prostatic hyperplasia (BPH), for example. According to other embodiments, after the target tissue has been drawn inward using the vessel displacement arrangement 111, a treatment apparatus 110 placed in the renal artery 12 can be configured to ablate the target tissue using other ablation mechanisms, such as mechanical (e.g., compression), electrical, optical, acoustic, pharmacological (e.g., delivery of a neurotoxin or venom), disruptive lysis, elelctroporation (“no heat”), or denaturation, for example.

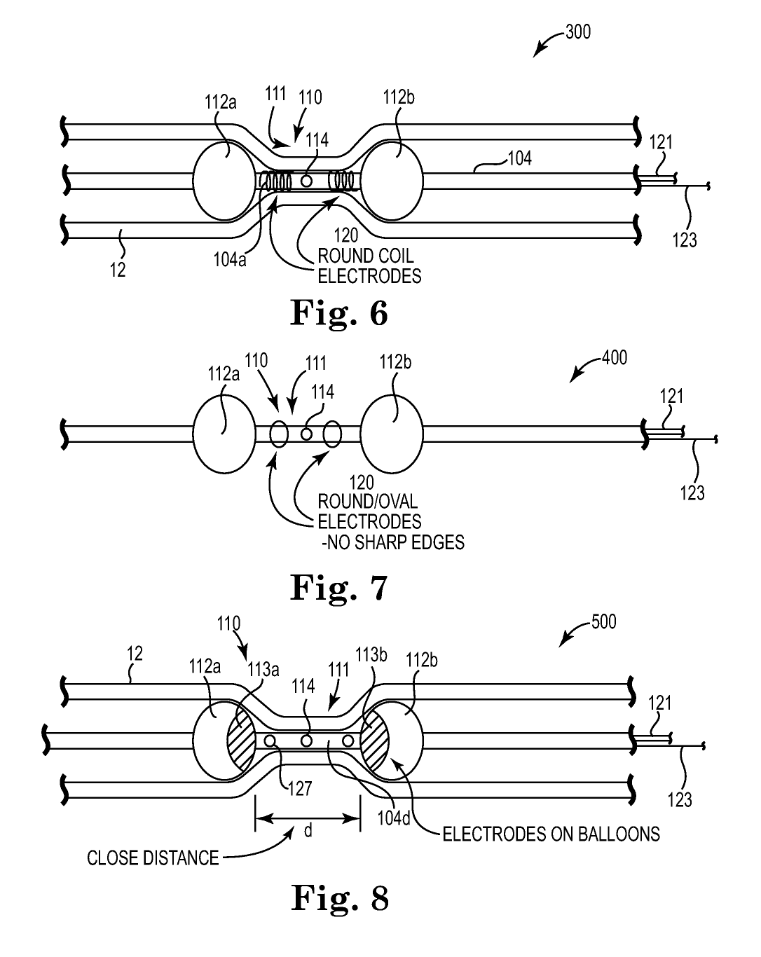

According to the embodiment shown in FIG. 5, the vessel displacement arrangement 111 situated at the distal end of catheter 200 incorporates a pair of electrodes 120 provided on a portion 104a of the catheter shaft 104 situated between the balloons 112a and 112b. The vessel displacement of arrangement 111 also includes a suction port 114 and a vacuum lumen 105 operable to draw in an isolated segment of the renal artery 12 as discussed above. The electrodes 120, according to various embodiments, form a bipolar electrode pair. In other embodiments, a single electrode 120 or multiple tied electrodes 120 can be used for operation in a unipolar mode, in which case an external return electrode, such as a pad electrode, is employed. Electrodes 120 are electrically coupled to a conductor arrangement 123 which extends to the proximal end of the catheter shaft 104. The conductor arrangement 123 is electrically coupled to a high frequency AC generator, such as an RF generator.

In some embodiments, the electrodes 120 can be driven in multiple modes, such as a stimulation mode and an ablation mode. With occlusion balloons 112a and 112b inflated to isolate a segment of the renal artery 12, suction is generated at the suction port 114 which aspirates blood trapped within the isolated segment and draws the artery wall into abutment with the section 104a of the catheter shaft 104. The electrodes 120 can be driven in a stimulation mode to aid in drawing the artery wall inward, such as by temporarily inducing arterial spasm. With the target nerve tissue drawn towards the arrangement of electrodes 120, RF energy is delivered to the electrodes 124 ablate the target nerve tissue.

In some embodiments, it may be desirable to provide cooling to the isolated segment of the renal artery 12 subject to RF ablation. Such embodiments can include one or both of a phase-change mechanism and a fluid circulation heat exchange arrangement of a type previously described. Artery cooling can be provided by cooling the occlusion balloons 112a and 112b, the section 104A of the catheter shaft 104 situated between the occlusion balloons 112a and 112b, or both apparatuses. It is noted that in embodiments that include ablation arrangements other than RF electrodes, an arrangement of electrodes 120 may be included for purposes of aiding the process of drawing the isolated artery segment into contact or near contact with an ablation arrangement (e.g., a cryothermal, acoustic, mechanical, or optical ablation arrangement). Accordingly, cryothermal cooling, RF stimulation and/or ablation, and other forms of ablation described herein may be used in a variety of combinations to provide for enhanced renal denervation. For example, a cryothermal element may be used in combination with an RF ablation arrangement, with one or both of the cryothermal cooling element and RF ablation arrangement delivering ablation therapy (e.g., freeze/thaw/heating cycling).

In the embodiment shown in FIG. 6, the vessel displacement arrangement 111 of catheter 300 incorporates a pair of round coil electrodes 120 situated between the balloons 112a and 112b. The vessel displacement of arrangement 111 also includes a suction port 114 and a vacuum lumen 105 operable to draw in an isolated segment of the renal artery 12 as previously discussed. The coil electrodes 120, according to various embodiments, form a bipolar electrode pair. In other embodiments, a single coil electrode 120 or multiple tied coil electrodes 120 can be used for operation in a unipolar mode. Coil electrodes 120 are electrically coupled to a conductor arrangement 123, which extends to the proximal end of the catheter shaft 104 and is electrically coupled to a high frequency AC generator, such as an RF generator.

According to the embodiment shown in FIG. 7, the vessel displacement arrangement 111 of catheter 400 incorporates a pair of round or oval electrodes 120 devoid of sharp edges situated between the balloons 112a and 112b. The vessel displacement of arrangement 111 also includes a suction port 114 and a vacuum lumen 105 operable to draw in an isolated segment of the renal artery 12 as previously discussed. The round or oval electrodes 120 can form a bipolar electrode pair or be configured as a unipolar electrode. The round or oval electrodes 120 are electrically coupled to a conductor arrangement 123, which extends to the proximal end of the catheter shaft 104 and is electrically coupled to a high frequency AC generator, such as an RF generator.

The coil electrodes 120 shown in FIG. 6 and the round/oval electrodes 120 shown in FIG. 7 can be driven in multiple modes, such as a stimulation mode and an ablation mode. Various embodiments can include one or both of a phase-change mechanism and a fluid circulation heat exchange arrangement of a type previously described.

FIG. 8 shows a further embodiment of a vessel displacement arrangement 111. In the embodiment shown in FIG. 8, the vessel displacement arrangement 111 of catheter 500 incorporates a treatment arrangement 110 which includes electrodes 113a and 113b respectively provided on inward facing surfaces of the balloons 112a and 112b. The electrode configuration shown in FIG. 8 serves to focus RF energy at the vessel wall segment held against the catheter section 104a by the suction created at the suction port 114 of the vessel displacement arrangement 111. Provision of the electrodes 113a and 113b on inward facing surfaces of the balloons 112a and 112b also serves to limit the extent of thermal damage to non-targeted tissue (i.e., tissue not captured between the balloons 112a and 112b). The distance, d, between the balloons 112a and 112b implemented in accordance with the embodiment shown in FIG. 8 is preferably about 4 or 5 mm, but can range between about 4 and 6 mm. The electrodes 113a and 113 shown in FIG. 8 may be formed using a variety of materials and techniques, including one or more metalized material layers and hydrophilic polymer material such as TECOPHILIC® or similar material that becomes electrically conductive when an electrolytic fluid is absorbed, for example.

According to various embodiments, the balloons 112a and 112b can incorporate one or several fluid conductive regions formed of a hydrophilic polymer. The regions 113a and 113b may define a single region or a multiplicity of such regions. The balloons 112a and 112b can be inflated using an electrically conductive fluid. The fluid conductive regions that define hydrophilic areas quickly absorb a water-based electrolytic solution (e.g., saline) to become conductive to RF electric current. TECOPHILIC® or TECOGEL® polyurethanes or similar materials (e.g., thermoplastic polyurethanes) can be used to form the hydrophilic areas of the balloons 112a and 112b, for example, which absorb significant amounts of a water-based electrolyte and quickly become electrically conductive when wet. In some configurations, the balloons 112a and 112b can include a first material that comprises a non-conductive polymer material, and the one or more fluid conductive regions provided on the balloons 112a and 112b each comprise a conductive thermoplastic polyurethane.

The hydrophilic polymer at the fluid conductive regions becomes electrically conductive in response to absorption of the conductive liquid used to pressurize the balloons 112a and 112b. In some embodiments, the balloons 112a and 112b are pressurized to a pressure (P1) greater than a pressure (P2) surrounding the balloons 112a and 112b. The pressure (P2) surrounding the balloon 102 may be the pressure of blood passing within the renal artery 12, for example. By way of further example, the pressure (P2) surrounding the balloons 112a and 112b may be the pressure of tissue at a treatment location of the body that exerts a force against the body 103 of the balloons 112a and 112b.

A pressure differential developed by pressurizing the balloons 112a and 112b to a pressure greater than that surrounding the balloons 112a and 112b facilitates perfusion of the conductive liquid through the fluid conductive regions of the balloons 112a and 112b and into a gap between an exterior surface of the balloons 112a and 112b and the inner wall of the renal artery 12. In this scenario, when the fluid conductive regions are energized using an RF power source, RF energy is conducted through the wall of the balloons 112a and 112b and to the artery wall via the fluid conductive regions and conductive fluid perfusion through the conducive regions. The RF energy conducted to the artery wall is preferably sufficient to ablate renal nerves included in the perivascular space adjacent the renal artery 12 or those located on or within the adventitia of the renal artery 12. Additional details of various balloon structures that incorporate hydrophilic material that can operate as RF electrodes in the manner discussed above are described in commonly owned U.S. Patent Publication No. ______________, filed as U.S. patent application Ser. No. 13/188,677 on Jul. 22, 2011, which is incorporated herein by reference.

In some embodiments, the shaft 104 of the catheter implementations discussed above may include one or more diversion lumens through which arterial blood may flow during application of suction to the artery wall. One or more inlet and outlet ports can be provided in the catheter shaft 104 at locations proximal and distal to the vessel deformation arrangement 111 or the treatment arrangement 110. The diversion lumen arrangement provides for continued blood flow through the renal artery 12 during the denervation procedure and may aid in cooling the artery tissue in contact with the treatment arrangement 110.

As was discussed previously, various types of ablation arrangements may be incorporated as part of the intravascular apparatus, including electrical, thermal, acoustic, mechanical, optical, lysic, neurotoxin, and pharmacological ablation arrangements, for example, or combinations of these arrangements. Details of various ultrasound denervation therapy apparatuses and methods that can be implemented in accordance with embodiments of the disclosure are described in commonly owned U.S. Patent Publication No. _______________, filed as U.S. patent application Ser. No. 13/086,116 on Apr. 13, 2011, which is incorporated herein by reference. Embodiments that utilize a laser or a high intensity flash lamp are also contemplated. Details of these and other phototherapy denervation therapy apparatuses and methods that can be implemented in accordance with embodiments of the disclosure are described in commonly owned U.S. Patent Publication No. ________________, filed as U.S. patent application Ser. No. 13/086,121 on Apr. 13, 2011, which is incorporated herein by reference.

Details of other denervation therapy apparatuses and methods that can be implemented in accordance with embodiments of the disclosure are described in commonly owned U.S. Patent Publication No. _________________, filed as U.S. patent application Ser. No. 12/980,952 on Dec. 29, 2010; U.S. Patent Publication No. _____________, filed as U.S. patent application Ser. No. 12/980,972 on Dec. 29, 2010; U.S. Patent Publication No. _________________, filed as U.S. patent application Ser. No. 13/157,844 on Jun. 10, 2011; U.S. Patent Publication No. _____________, filed as U.S. patent application Ser. No. 13/087,163 on Apr. 14, 2011; and U.S. Patent Publication No. ________________, filed as U.S. patent application Ser. No. 12/980,948 filed on Dec. 29, 2010; all of which are incorporated herein by reference.

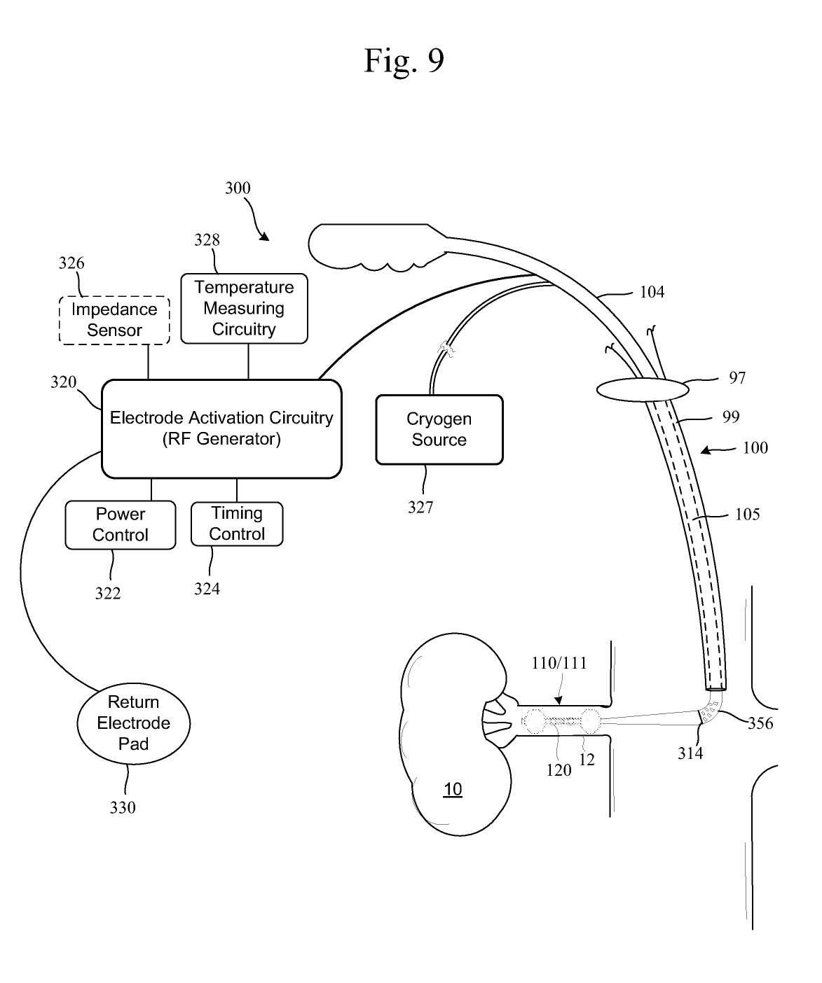

FIG. 9 shows a representative renal ablation apparatus 300 in accordance with various embodiments of the disclosure. Although the apparatus 300 is configured for RF ablation, it is understood that the apparatus 300 can be configured to deliver other forms of ablative energy, such as ultrasound, optical, mechanical, lysic, and cryothermal energy, for example, or any other form of ablative energy described herein. The apparatus 300 illustrated in FIG. 9 includes external electrode activation circuitry 320 which comprises power control circuitry 322 and timing control circuitry 324. The external electrode activation circuitry 320, which includes an RF generator, is coupled to temperature measuring circuitry 328 and may be coupled to an optional impedance sensor 326. The catheter 100 includes a shaft 104 that incorporates a lumen arrangement 105 which can be configured for receiving a variety of components, such as conductors, pharmacological agents, actuator elements, obturators, sensors, or other components as needed or desired. The catheter 100 can be delivered to the renal artery 12 using a guide sheath or guiding catheter 99 via a percutaneous access location 97. The catheter 100 may include a hinge mechanism 356 to aid in navigating the catheter around the nearly 90° turn from the aorta and into the renal artery 12.

The RF generator of the external electrode activation circuitry 320 may include a return pad electrode 330 that is configured to comfortably engage the patient's back or other portion of the body near the kidneys. In this configuration (unipolar), a single RF electrode 120 or multiple tied electrodes 120 may be situated at the distal end of the catheter 100. In a bipolar configuration, at least two RF electrodes 120 are incorporated as part of the treatment arrangement 110 and/or vessel deformation arrangement 111, in which case the return electrode pad 330 is not needed.

Radiofrequency energy produced by the RF generator is coupled to the electrodes 120 by a conductor arrangement disposed in the lumen arrangement 105 of the catheter shaft 104. The RF energy flows through the electrodes 120 in accordance with a predetermined activation sequence (e.g., sequential or concurrent) to ablate perivascular renal nerves adjacent the renal artery 12. In general, when renal artery tissue temperatures rise above about 113° F. (50° C.), protein is permanently damaged (including those of renal nerve fibers). If heated over about 65° C., collagen denatures and tissue shrinks. If heated over about 65° C. and up to 100° C., cell walls break and oil separates from water. Above about 100° C., tissue desiccates.

The electrode activation circuitry 320 is configured to control activation and deactivation of the electrodes 120 in accordance with a predetermined energy delivery protocol and in response to signals received from the temperature measuring circuitry 328, and/or the impedance sensor 336. The electrode activation circuitry 320 controls RF energy delivered to the electrodes 120 so as to maintain the current densities at a level sufficient to cause heating of the target tissue to at least a temperature of 55° C., for example. A cooling fluid dispensed by a cryogen source 327 may be delivered to the distal end of the catheter 100 to provide cooling at the electrode-tissue interface. The cyrogen source 327 may be controlled automatically by the electrode activation circuitry 320.

In some embodiments, temperature sensors are situated at the treatment arrangement 110 and/or vessel deformation arrangement 111 to provide for continuous monitoring of renal artery tissue temperatures, and RF generator power can be automatically adjusted so that the target temperatures are achieved and maintained. An impedance sensor arrangement 326 may be used to measure and monitor electrical impedance during RF denervation therapy, and the power and timing of the RF generator 320 may be moderated based on the impedance measurements or a combination of impedance and temperature measurements.

Marker bands 314 can be placed on one or multiple parts at the distal end of the catheter 100 to enable visualization during the procedure. Other portions of the catheter 100, such as one or more portions of the shaft 104 (e.g., at the hinge mechanism 356 and/or the treatment arrangement 110 and/or vessel deformation arrangement 111), may include a marker band 314. The marker bands 314 may be solid or split bands of platinum or other radiopaque metal, for example, to aid in catheter navigation and electrode positioning. A braid and/or electrodes or sensors of the catheter 100, according to some embodiments, can be radiopaque.

Various embodiments disclosed herein are generally described in the context of ablation of perivascular renal nerves for control of hypertension. It is understood, however, that embodiments of the disclosure have applicability in other contexts, such as performing ablation from within other vessels of the body, including other arteries, veins, and vessels (e.g., cardiac and urinary vasculature and vessels), and other tissues of the body, including various organs. For example, various embodiments may be configured to treat benign prostatic hyperplasia or to diagnose and/or treat a tumor using an appropriate medical device advanced to the treatment site through an appropriate body pathway. Embodiments of the disclosure can be implemented for use in a variety of ablation procedures involving cardiac vessels, such as for cardiac arrhythmia therapy, for example. Embodiments of the disclosure can be implemented to position other ablation devices in intimate contact with tissue, including positioning intravascular therapy devices, urological devices, devices in a heart chamber, devices in the gastrointestinal tract, among others.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. An apparatus, comprising:
a catheter comprising a flexible shaft having a proximal end, a distal end, a length, and a lumen arrangement extending between the proximal and distal ends, the length of the shaft sufficient to access a patient's renal artery relative to a percutaneous access location;
a balloon arrangement provided at the distal end of the catheter and configured for deployment within the renal artery, the balloon arrangement fluidly coupled to the lumen arrangement and comprising a distal balloon and a proximal balloon spaced apart from the distal balloon;
an ablation arrangement provided at the distal end of the catheter and configured to ablate perivascular renal nerves adjacent the renal artery; and
a port provided in a section of the shaft between the first and second balloons and fluidly coupled to the lumen arrangement, the port and the balloon arrangement configured to cooperatively draw a wall segment of the renal artery and perivascular renal nerves adjacent the renal artery wall segment inwardly into proximity or abutment with the ablation arrangement in response to a negative pressure developed at the port.

2. The apparatus of claim 1, wherein the ablation arrangement and the balloon arrangement define an integral apparatus.

3. The apparatus of claim 1, wherein at least a portion of the ablation arrangement is situated between the distal and proximal balloons.

4. The apparatus of claim 1, wherein the port and balloon arrangement cooperate to stabilize the renal artery wall segment in proximity or abutment with the ablation arrangement during ablation of the perivascular renal nerves.

5. The apparatus of claim 1, wherein:
the lumen arrangement comprises a vacuum lumen; and
the port and the vacuum lumen are configured to facilitate aspiration of blood trapped between the distal and proximal balloons.

6. The apparatus of claim 1, wherein the ablation arrangement is configured to create a circumferential lesion in the perivascular renal nerves.

7. The apparatus of claim 1, wherein the ablation arrangement comprises a cryothermal ablation arrangement.

8. The apparatus of claim 1, wherein the ablation arrangement comprises an ultrasound ablation arrangement.

9. The apparatus of claim 1, wherein:
the lumen arrangement comprises a supply lumen and an exhaust lumen;
each of the distal and proximal balloons is configured to receive a liquid phase cryogen from the supply lumen and return gas exhaust to the exhaust lumen resulting from a phase-change of the cryogen; and
the ablation arrangement comprises a zone of concentrated cooling produced by the cooled distal and proximal balloons.

10. The apparatus of claim 1, comprising at least one electrode situated at or near at least one of the distal and proximal balloons, the electrode electrically coupled to one or more electrical conductors extending along the shaft of the catheter, the at least one electrode configured to deliver stimulation energy sufficient to cause relaxation of the renal artery wall segment, thereby enhancing drawing of the renal artery wall segment and the perivascular renal nerves inwardly into proximity or abutment with the ablation arrangement.

11. The apparatus of claim 1, wherein the ablation arrangement comprises an electrode arrangement electrically coupled to one or more electrical conductors extending along the shaft of the catheter, the electrode arrangement configured to deliver high frequency AC current to the perivascular renal nerves.

12. The apparatus of claim 11, wherein the electrode arrangement comprises at least one pair of electrodes arranged in a bipolar configuration.

13. The apparatus of claim 11, wherein the electrode arrangement comprises one or more of annular electrodes, round electrodes, and oval electrodes.

13. The apparatus of claim 11, wherein the electrode arrangement comprises one or more coil electrodes.

14. The apparatus of claim 11, further comprising a cooling arrangement configured to provide cooling to the renal artery wall segment.

15. The apparatus of claim 1, wherein each of the distal and proximal balloons comprises a region defining one or more electrodes.

16. The apparatus of claim 1, wherein each of the distal and proximal balloons comprises a port facing region comprising a hydrophilic polymer that becomes electrically conductive in response to absorption of a conductive liquid.

17. The apparatus of claim 1, wherein the ablation arrangement comprises one or a combination of a thermal, electrical, acoustic, optical, mechanical, lysic, neurotoxin or pharmacological ablation apparatus.

18. An apparatus, comprising:
a catheter;
a balloon arrangement provided at a distal end of the catheter and configured for deployment within a vessel, the balloon arrangement comprising a distal balloon and a proximal balloon spaced apart from the distal balloon;
an ablation arrangement provided at the distal end of the catheter and configured to ablate target tissue proximate the vessel; and
a vessel deformation arrangement comprising at least a portion of the balloon arrangement and configured to draw a wall segment of the vessel and the target tissue inwardly towards the ablation arrangement.

19. The apparatus of claim 18, wherein the vessel deformation arrangement comprises a port provided in a section of the shaft between the distal and proximal balloons and fluidly coupled to a vacuum lumen of the catheter, the port and balloon arrangement configured to cooperatively draw the wall segment of the vessel and the target tissue inwardly towards the ablation arrangement in response to a negative pressure developed at the port.

20. The apparatus of claim 18, wherein the ablation arrangement comprises one or a combination of a cryothermal, electrical, acoustic, optical, mechanical, lysic, neurotoxin or pharmacological ablation apparatus.

21. A method, comprising:
drawing a wall segment of a vessel and target tissue adjacent the vessel inwardly towards an ablation arrangement deployed in the vessel;
with the vessel wall segment and target tissue inwardly drawn into contact or proximity with the ablation arrangement, ablating the target tissue; and
terminating ablation of the target tissue and drawing of the vessel wall segment, thereby allowing the diameter of the vessel wall segment to return to its original size.

22. The method of claim 21, wherein the vessel comprises a renal artery, and the target tissue comprises perivascular renal nerves adjacent the renal artery.
